(19) United States
(12) Patent Application Publication
(10) Pub. No.: US 2025/0057603 A1
(43) Pub. Date: Feb. 20, 2025

(54) SYSTEMS AND METHODS FOR REAL-TIME VISUALIZATION OF ANATOMY IN NAVIGATED PROCEDURES

(71) Applicant: Medtronic Navigation, Inc., Lafayette, CO (US)

(72) Inventors: Nikhil Mahendra, Broomfield, CO (US); Victor D. Snyder, Erie, CO (US); Jinglin Liu, Westminster, CO (US)

(21) Appl. No.: 18/787,810

(22) Filed: Jul. 29, 2024

Related U.S. Application Data
(60) Provisional application No. 63/532,974, filed on Aug. 16, 2023.

(51) Int. Cl. A61B 34/20; A61B 17/16; A61B 34/30

(52) U.S. Cl. CPC A61B 34/20; A61B 17/16; A61B 34/30; A61B 2034/2065; A61B 2034/2074

(57) ABSTRACT

A system according to an embodiment of the present disclosure includes: a processor; and a memory storing data thereon that, when processed by the processor, enable the processor to: receive an image depicting an anatomical element; segment the image into a segmented image that includes a plurality of voxels; track a portion of a surgical instrument as the portion of the surgical instrument interacts with the anatomical element; identify, based on the tracking, an area from the segmented image representative of a section of the anatomical element that interacts with the portion of the surgical instrument; modify one or more voxels from the plurality of voxels that reside within the area identified from the segmented image as being representative of the section of the anatomical element that interacts with the portion of the surgical instrument; and render, to a display, the segmented image showing the modified one or more voxels.

### CROSS-REFERENCE TO RELATED APPLICATION

This application claims the benefit of and priority to U.S. Provisional Patent Application No. 63/532,974 filed Aug. 16, 2023, the entire disclosure of which is incorporated by reference herein.

### BACKGROUND

The present disclosure is generally directed to surgical navigation, and relates more particularly to visualization of anatomy during navigated surgeries or surgical procedures.

Surgical robots may assist a surgeon or other medical provider in carrying out a surgical procedure, or may complete one or more surgical procedures autonomously. Imaging may be used by a medical provider for diagnostic and/or therapeutic purposes. Patient anatomy can change over time, particularly following placement of a medical implant in the patient anatomy.

### BRIEF SUMMARY

Example aspects of the present disclosure include:

A system according to at least one embodiment of the present disclosure comprises: a processor; and a memory storing data thereon that, when processed by the processor, enable the processor to: receive an image depicting an anatomical element; segment the image into a segmented image that includes a plurality of voxels; track a portion of a surgical instrument as the portion of the surgical instrument interacts with the anatomical element; identify, based on the tracking, an area from the segmented image representative of a section of the anatomical element that interacts with the portion of the surgical instrument; modify one or more voxels from the plurality of voxels that reside within the area identified from the segmented image as being representative of the section of the anatomical element that interacts with the portion of the surgical instrument; and render, to a display, the segmented image showing the modified one or more voxels.

Any of the features herein, wherein the one or more voxels are rendered with a first visual depiction a first time, and wherein the one or more voxels are rendered with a second visual depiction at a second time later than the first time.

Any of the features herein, wherein the modified one or more voxels are rendered with at least one of a different color and a different border than the plurality of voxels.

Any of the features herein, wherein the portion of the surgical instrument is capable of resecting anatomical tissue.

Any of the features herein, wherein the image is a two-dimensional image or a three-dimensional image.

Any of the features herein, wherein the tracking comprises determining a pose of the portion of the surgical instrument relative to the anatomical element as the portion of the surgical instrument interacts with the anatomical element.

Any of the features herein, wherein the modified one or more voxels indicate that the portion of the anatomical element has been resected.

A system according to at least one embodiment of the present disclosure comprises: a processor; and a memory coupled with the processor and storing data thereon that, when processed by the processor, enable the processor to: receive a segmented image depicting an anatomical element segmented into a plurality of voxels; render, to a display, the segmented image; track a surgical tool as the surgical tool interacts with the anatomical element; determine, based on the tracking, a voxel of the plurality of voxels representative of a portion of the anatomical element that interacts with the surgical tool; and update a visual depiction of the voxel shown in the segmented image on the display.

Any of the features herein, wherein the update of the visual depiction of the voxel comprises a change in at least one of a color and a border of the voxel.

Any of the features herein, wherein the update of the visual depiction of the voxel comprises an indicator that the portion of the anatomical element has been resected.

Any of the features herein, wherein the segmented image comprises a two-dimensional image or a three-dimensional image.

Any of the features herein, wherein the segmented image is received from an artificial intelligence data model.

Any of the features herein, wherein the artificial intelligence data model comprises a convolutional neural network that receives image data as an input and outputs the segmented image.

Any of the features herein, wherein the tracking comprises determining a pose of the surgical tool relative to the anatomical element as the surgical tool interacts with the anatomical element.

Any of the features herein, wherein the update of the visual depiction of the voxel is based on at least one of a type of surgical tool and a surgical workflow.

A system according to at least one embodiment of the present disclosure comprises: a processor; and a memory coupled with the processor and storing data thereon that, when processed by the processor, enable the processor to: receive image data associated with an anatomical element; segment the image data into a plurality of voxels; render, to a display, a visual depiction of the plurality of voxels; track an operative portion of a surgical instrument as the operative portion of the surgical instrument interacts with the anatomical element; identify, based on the tracking, a voxel of the plurality of voxels associated with the operative portion of the surgical instrument; and render, based on the tracking, an updated visual depiction of the image data that includes a modified version of the voxel.

Any of the features herein, wherein the operative portion of the surgical instrument is capable of resecting anatomical tissue.

Any of the features herein, wherein the modified version of the voxel provides an indicator that a section of the anatomical element has been resected.

Any of the features herein, wherein the modified version of the voxel is rendered in the updated visual depiction with a first visual indicator when the surgical instrument is a first type of surgical instrument, and wherein the voxel is rendered with a second visual indicator when the surgical instrument is a second type of surgical instrument.

Any of the features herein, wherein the first visual indicator indicates that a section of the anatomical element has been resected, and wherein the second visual indicator indicates that the section of the anatomical element has been decorticated.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/embodiments in combination with any one or more other aspects/features/embodiments.

Use of any one or more of the aspects or features as disclosed herein.

It is to be appreciated that any feature described herein can be claimed in combination with any other feature(s) as described herein, regardless of whether the features come from the same described embodiment.

The details of one or more aspects of the disclosure are set forth in the accompanying drawings and the description below. Other features, objects, and advantages of the techniques described in this disclosure will be apparent from the description and drawings, and from the claims.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X1-Xn, Y1-Ym, and Z1-Zo, the phrase is intended to refer to a single element selected from X, Y, and Z, a combination of elements selected from the same class (e.g., X1 and X2) as well as a combination of elements selected from two or more classes (e.g., Y1 and Zo).

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages of the present disclosure will become apparent to those skilled in the art upon consideration of the embodiment descriptions provided hereinbelow.

### BRIEF DESCRIPTION OF THE SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 is a block diagram of a system according to at least one embodiment of the present disclosure;

FIG. 2A is a diagram of the surgical tool positioned proximate an anatomical element according to at least one embodiment of the present disclosure;

FIG. 2B is a diagram of the surgical tool moving relative to the anatomical element according to at least one embodiment of the present disclosure;

FIG. 2C is a diagram of the surgical tool moving relative to the anatomical element according to at least one embodiment of the present disclosure;

FIG. 2D is a depiction of segmented voxels of the anatomical element according to at least one embodiment of the present disclosure;

FIG. 2E is a depiction of segmented voxels of the anatomical element according to at least one embodiment of the present disclosure; and

FIG. 3 is a flowchart according to at least one embodiment of the present disclosure.

### DETAILED DESCRIPTION

It should be understood that various aspects disclosed herein may be combined in different combinations than the combinations specifically presented in the description and accompanying drawings. It should also be understood that, depending on the example or embodiment, certain acts or events of any of the processes or methods described herein may be performed in a different sequence, and/or may be added, merged, or left out altogether (e.g., all described acts or events may not be necessary to carry out the disclosed techniques according to different embodiments of the present disclosure). In addition, while certain aspects of this disclosure are described as being performed by a single module or unit for purposes of clarity, it should be understood that the techniques of this disclosure may be performed by a combination of units or modules associated with, for example, a computing device and/or a medical device.

In one or more examples, the described methods, processes, and techniques may be implemented in hardware, software, firmware, or any combination thereof. If implemented in software, the functions may be stored as one or more instructions or code on a computer-readable medium and executed by a hardware-based processing unit. Alternatively or additionally, functions may be implemented using machine learning models, neural networks, artificial neural networks, or combinations thereof (alone or in combination with instructions). Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors (e.g., Intel Core i3, i5, i7, or i9 processors; Intel Celeron processors; Intel Xeon processors; Intel Pentium processors; AMD Ryzen processors; AMD Athlon processors; AMD Phenom processors; Apple A10 or 10× Fusion processors; Apple A11, A12, A12X, A12Z, or A13 Bionic processors; or any other general purpose microprocessors), graphics processing units (e.g., Nvidia GeForce RTX 2000-series processors, Nvidia GeForce RTX 3000-series processors, AMD Radeon RX 5000-series processors, AMD Radeon RX 6000-series processors, or any other graphics processing units), application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

Before any embodiments of the disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

In spinal fusion procedures, preparation for spinal decompression can involve removal of some bony anatomy, such as lamina, facet joints, bone spurs, and/or the like. For example, during facet decortication for enabling posterior fusion, partial layers of bone on the surfaces of posterior sections of a vertebra are removed before adding bone graft. As another example, while placing pedicle and cortical screws, a pilot hole in a vertebra can be created with surgical instruments (e.g., a drill, such as a Midas, or an awl), and threads can be created with instruments such as a tap. Such examples involve resecting bone from vertebrae, but the depiction of the vertebrae on the navigation screen is not updated without new imaging.

In some embodiments, navigation is used to identify vertebral bone that has been removed during a surgery or surgical procedure, such as a spine surgery. Based on prior information about the surgical workflow and the surgical tools used within the surgical workflow, the state of the vertebral anatomy (e.g., the state of a vertebra after facet joint resection) can be updated on the navigation screen in real-time without the need for new intraoperative imaging.

Navigation or robotics may assume that the spine is rigid, with the goal of spinal surgery being altering the spine's shape. However, the accuracy of the navigation or robotics decreases as the spine is manipulated. Conventional art methods include workflow modifications, rescanning, reregistration, and guessing. What is missing are techniques for objectively understanding anatomical correction intraoperatively. Segmental tracking updates clinical images based on real-time knowledge of each vertebra's unique position and orientation, maintaining accuracy and enabling intraoperative assessment of anatomical correction.

In some examples, segmental tracking and tracked instruments such as taps and drills can be used to update the visualization of the vertebrae to show resected bony voxels of pilot holes, tapped screw holes, combinations thereof, and/or the like. In other examples, segmental tracking and tracked instruments such as drills (e.g., Midas MR8™ drills, Midas Rex™ Mazor™ Facet Decortication Acorn Tool), voxels of vertebrae where a drill bit has been used to decorticate facet/lamina can be visualized with a different color to indicate an updated vertebral anatomy state. In some examples, information about an instrument's tip position (e.g., when the instrument is a tracked instrument such as an osteotome) in conjunction with image processing methods such as connected components analysis can be used to find resected bony anatomy such as facet joints and update visualization.

Embodiments of the present disclosure provide technical solutions to one or more of the problems of (1) inaccurate visual depictions of anatomical elements during surgeries or surgical procedures and (2) additional or excessive radiation exposure due to additional intraoperative imaging.

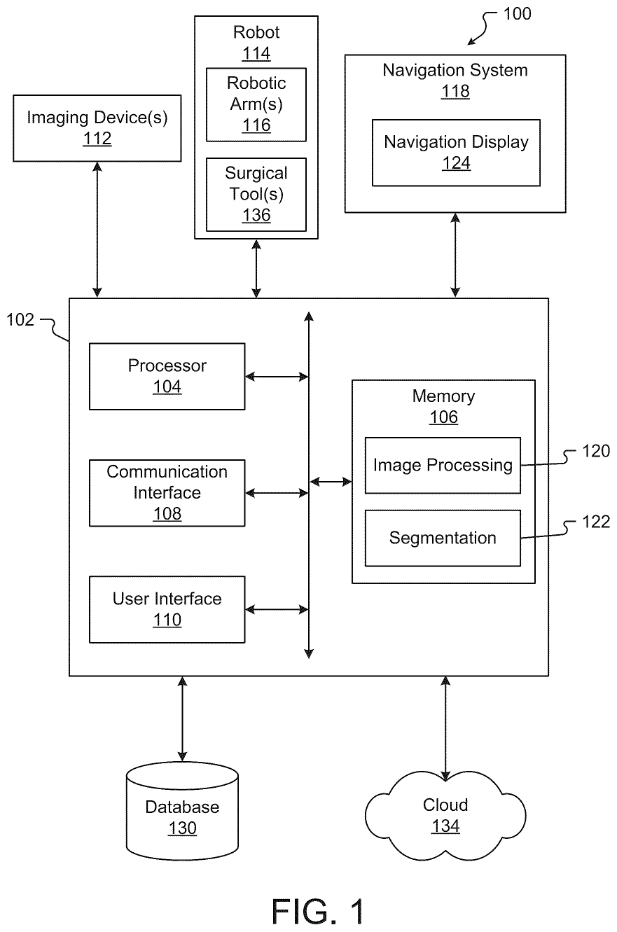

Turning first to FIG. 1, a block diagram of a system 100 according to at least one embodiment of the present disclosure is shown. The system 100 may be used to navigate surgical tools and/or image anatomical elements during surgical procedures; to update anatomical element visualization to account for changes in the state of anatomical element(s) during a surgery or surgical procedure; to control, pose, and/or otherwise manipulate a surgical mount system, a surgical arm, and/or surgical tools attached thereto; and/or carry out one or more other aspects of one or more of the methods disclosed herein. The system 100 comprises a computing device 102, one or more imaging devices 112, a robot 114, a navigation system 118, a database 130, and/or a cloud or other network 134. Systems according to other embodiments of the present disclosure may comprise more or fewer components than the system 100. For example, the system 100 may not include one or more components of the computing device 102, the database 130, and/or the cloud 134.

The computing device 102 comprises a processor 104, a memory 106, a communication interface 108, and a user interface 110. Computing devices according to other embodiments of the present disclosure may comprise more or fewer components than the computing device 102.

The processor 104 of the computing device 102 may be any processor described herein or any similar processor. The processor 104 may be configured to execute instructions or other data stored in the memory 106, which instructions or data may cause the processor 104 to carry out one or more computing steps utilizing or based on data received from the imaging device 112, the robot 114, the navigation system 118, the database 130, and/or the cloud 134.

The memory 106 may be or comprise RAM, DRAM, SDRAM, other solid-state memory, any memory described herein, or any other tangible, non-transitory memory for storing computer-readable data and/or instructions. The memory 106 may store information or data useful for completing, for example, any step of the method 300 described herein, or of any other methods. The memory 106 may store, for example, instructions and/or machine learning models that support one or more functions of the robot 114. For instance, the memory 106 may store content (e.g., instructions and/or machine learning models) that, when executed by the processor 104, enable image processing 120 and/or segmentation 122. Such content, if provided as in instruction, may, in some embodiments, be organized into one or more applications, modules, packages, layers, or engines. Alternatively or additionally, the memory 106 may store other types of content or data (e.g., machine learning models, artificial neural networks, deep neural networks, etc.) that can be processed by the processor 104 to carry out the various method and features described herein. Thus, although various contents of memory 106 may be described as instructions, it should be appreciated that functionality described herein can be achieved through use of instructions, algorithms, and/or machine learning models. The data, algorithms, and/or instructions may cause the processor 104 to manipulate data stored in the memory 106 and/or received from or via the imaging device 112, the robot 114, the database 130, and/or the cloud 134.

The computing device 102 may also comprise a communication interface 108. The communication interface 108 may be used for receiving image data or other information from an external source (such as the imaging device 112, the robot 114, the navigation system 118, the database 130, the cloud 134, and/or any other system or component not part of the system 100), and/or for transmitting instructions, images, or other information to an external system or device (e.g., another computing device 102, the imaging device 112, the robot 114, the navigation system 118, the database 130, the cloud 134, and/or any other system or component not part of the system 100). The communication interface 108 may comprise one or more wired interfaces (e.g., a USB port, an Ethernet port, a Firewire port) and/or one or more wireless transceivers or interfaces (configured, for example, to transmit and/or receive information via one or more wireless communication protocols such as 802.11a/b/g/n, Bluetooth, NFC, ZigBee, and so forth). In some embodiments, the communication interface 108 may be useful for enabling the device 102 to communicate with one or more other processors 104 or computing devices 102, whether to reduce the time needed to accomplish a computing-intensive task or for any other reason.

The computing device 102 may also comprise one or more user interfaces 110. The user interface 110 may be or comprise a keyboard, mouse, trackball, monitor, television, screen, touchscreen, and/or any other device for receiving information from a user and/or for providing information to a user. The user interface 110 may be used, for example, to receive a user selection or other user input regarding any step of any method described herein. Notwithstanding the foregoing, any required input for any step of any method described herein may be generated automatically by the system 100 (e.g., by the processor 104 or another component of the system 100) or received by the system 100 from a source external to the system 100. In some embodiments, the user interface 110 may be useful to allow a surgeon or other user to modify instructions to be executed by the processor 104 according to one or more embodiments of the present disclosure, and/or to modify or adjust a setting of other information displayed on the user interface 110 or corresponding thereto.

Although the user interface 110 is shown as part of the computing device 102, in some embodiments, the computing device 102 may utilize a user interface 110 that is housed separately from one or more remaining components of the computing device 102. In some embodiments, the user interface 110 may be located proximate one or more other components of the computing device 102, while in other embodiments, the user interface 110 may be located remotely from one or more other components of the computer device 102.

The imaging device 112 may be operable to image anatomical feature(s) (e.g., a bone, veins, tissue, etc.) and/or other aspects of patient anatomy to yield image data (e.g., image data depicting or corresponding to a bone, veins, tissue, etc.). “Image data” as used herein refers to the data generated or captured by an imaging device 112, including in a machine-readable form, a graphical/visual form, and in any other form. In various examples, the image data may comprise data corresponding to an anatomical feature of a patient, or to a portion thereof. The image data may be or comprise a preoperative image, an intraoperative image, a postoperative image, or an image taken independently of any surgical procedure. In some embodiments, a first imaging device 112 may be used to obtain first image data (e.g., a first image) at a first time, and a second imaging device 112 may be used to obtain second image data (e.g., a second image) at a second time after the first time. The imaging device 112 may be capable of taking a two-dimensional (2D) image or a three-dimensional (3D) image to yield the image data. The imaging device 112 may be or comprise, for example, an ultrasound scanner (which may comprise, for example, a physically separate transducer and receiver, or a single ultrasound transceiver), an O-arm, a C-arm, a G-arm, or any other device utilizing X-ray-based imaging (e.g., a fluoroscope, a CT scanner, or other X-ray machine), a magnetic resonance imaging (MRI) scanner, an optical coherence tomography (OCT) scanner, an endoscope, a microscope, an optical camera, a thermographic camera (e.g., an infrared camera), a radar system (which may comprise, for example, a transmitter, a receiver, a processor, and one or more antennae), or any other imaging device 112 suitable for obtaining images of an anatomical feature of a patient. The imaging device 112 may be contained entirely within a single housing, or may comprise a transmitter/emitter and a receiver/detector that are in separate housings or are otherwise physically separated.

In some embodiments, the imaging device 112 may comprise more than one imaging device 112. For example, a first imaging device may provide first image data and/or a first image, and a second imaging device may provide second image data and/or a second image. In still other embodiments, the same imaging device may be used to provide both the first image data and the second image data, and/or any other image data described herein. The imaging device 112 may be operable to generate a stream of image data. For example, the imaging device 112 may be configured to operate with an open shutter, or with a shutter that continuously alternates between open and shut so as to capture successive images. For purposes of the present disclosure, unless specified otherwise, image data may be considered to be continuous and/or provided as an image data stream if the image data represents two or more frames per second.

The robot 114 may be any surgical robot or surgical robotic system. The robot 114 may be or comprise, for example, the Mazor X™ Stealth Edition robotic guidance system. The robot 114 may be configured to position the imaging device 112 at one or more precise position(s) and orientation(s), and/or to return the imaging device 112 to the same position(s) and orientation(s) at a later point in time. The robot 114 may additionally or alternatively be configured to manipulate a surgical tool (whether based on guidance from the navigation system 118 or not) to accomplish or to assist with a surgical task. In some embodiments, the robot 114 may be configured to hold and/or manipulate an anatomical element during or in connection with a surgical procedure. The robot 114 may comprise one or more robotic arms 116. In some embodiments, the robotic arm 116 may comprise a first robotic arm and a second robotic arm, though the robot 114 may comprise more than two robotic arms. In some embodiments, one or more of the robotic arms 116 may be used to hold and/or maneuver the imaging device 112. In embodiments where the imaging device 112 comprises two or more physically separate components (e.g., a transmitter and receiver), one robotic arm 116 may hold one such component, and another robotic arm 116 may hold another such component. Each robotic arm 116 may be positionable independently of the other robotic arm. The robotic arms 116 may be controlled in a single, shared coordinate space, or in separate coordinate spaces.

The robot 114, together with the robotic arm 116, may have, for example, one, two, three, four, five, six, seven, or more degrees of freedom. Further, the robotic arm 116 may be positioned or positionable in any pose, plane, and/or focal point. The pose includes a position and an orientation. As a result, an imaging device 112, surgical tool, or other object held by the robot 114 (or, more specifically, by the robotic arm 116) may be precisely positionable in one or more needed and specific positions and orientations.

The robotic arm(s) 116 may comprise one or more sensors that enable the processor 104 (or a processor of the robot 114) to determine a precise pose in space of the robotic arm 116 (as well as any object or element held by or secured to the robotic arm 116).

In some embodiments, reference markers (e.g., navigation markers) may be placed on the robot 114 (including, e.g., on the robotic arm 116), the imaging device 112, or any other object in the surgical space. The reference markers may be tracked by the navigation system 118, and the results of the tracking may be used by the robot 114 and/or by an operator of the system 100 or any component thereof. In some embodiments, the navigation system 118 can be used to track other components of the system (e.g., imaging device 112) and the system can operate without the use of the robot 114 (e.g., with the surgeon manually manipulating the imaging device 112 and/or one or more surgical tools, based on information and/or instructions generated by the navigation system 118, for example).

The navigation system 118 may provide navigation for a surgeon and/or a surgical robot during an operation. The navigation system 118 may be any now-known or future-developed navigation system, including, for example, the Medtronic StealthStation™ S8 surgical navigation system or any successor thereof. The navigation system 118 may include one or more cameras or other sensor(s) for tracking one or more reference markers, navigated trackers, or other objects within the operating room or other room in which some or all of the system 100 is located. The one or more cameras may be optical cameras, infrared cameras, or other cameras. In some embodiments, the navigation system 118 may comprise one or more electromagnetic sensors. In various embodiments, the navigation system 118 may be used to track a position and orientation (e.g., a pose) of the imaging device 112, the robot 114 and/or robotic arm 116, and/or one or more surgical tools (or, more particularly, to track a pose of a navigated tracker attached, directly or indirectly, in fixed relation to the one or more of the foregoing).

The navigation system 118 may include or be connected to a navigation display 124 (e.g., navigation display 124) for displaying one or more images from an external source (e.g., the computing device 102, imaging device 112, or other source) or for displaying an image and/or video stream from the one or more cameras or other sensors of the navigation system 118. In some examples, the navigation display 124 may be similar to or the same as the user interface 110, and may communicate with one or more other components of the system 100 (e.g., via the communication interface 108, via the cloud 134, etc.). The navigation system 118 may be configured to provide guidance to a surgeon or other user of the system 100 or a component thereof, to the robot 114, or to any other element of the system 100 regarding, for example, a pose of one or more anatomical elements, whether or not a tool is in the proper trajectory, and/or how to move a tool into the proper trajectory to carry out a surgical task according to a preoperative or other surgical plan. Such guidance may be provided on the navigation display 124. The navigation display 124 is also configured to be updated with modified depictions of patient anatomy through out a step, portion of, or the entirety of the surgery or surgical procedure, as discussed in further detail below.

The database 130 may store information that correlates one coordinate system to another (e.g., one or more robotic coordinate systems to a patient coordinate system and/or to a navigation coordinate system). The database 130 may additionally or alternatively store, for example, one or more surgical plans (including, for example, pose information about a target and/or image information about a patient's anatomy at and/or proximate the surgical site, for use by the robot 114, the navigation system 118, and/or a user of the computing device 102 or of the system 100); one or more images useful in connection with a surgery to be completed by or with the assistance of one or more other components of the system 100; and/or any other useful information. The database 130 may be configured to provide any such information to the computing device 102 or to any other device of the system 100 or external to the system 100, whether directly or via the cloud 134. In some embodiments, the database 130 may be or comprise part of a hospital image storage system, such as a picture archiving and communication system (PACS), a health information system (HIS), and/or another system for collecting, storing, managing, and/or transmitting electronic medical records including image data.

The cloud 134 may be or represent the Internet or any other wide area network. The computing device 102 may be connected to the cloud 134 via the communication interface 108, using a wired connection, a wireless connection, or both. In some embodiments, the computing device 102 may communicate with the database 130 and/or an external device (e.g., a computing device) via the cloud 134.

The system 100 comprises a surgical tool 136. The surgical tool 136 may be configured to drill, burr, mill, cut, saw, ream, tap, etc. into anatomical tissues such as patient anatomy (e.g., soft tissues, bone, etc.). In some embodiments, the system 100 may comprise multiple surgical tools, with each surgical tool performing a different surgical task (e.g., a surgical drill for drilling, a surgical mill for milling, an osteotome for cutting bone, etc.). In other embodiments, the surgical tool 136 may provide an adapter interface to which different working ends can be attached to perform multiple different types of surgical maneuvers (e.g., the surgical tool 136 may be able to receive one or more different tool bits, such that the surgical tool 136 can drill, mill, cut, saw, ream, tap, etc. depending on the tool bit coupled with the surgical tool 136). The surgical tool 136 may be operated autonomously or semi-autonomously.

In some embodiments, the surgical tool 136 may be attached to a robotic arm 116, such that movement of the robotic arm 116 correspondingly causes movement in the surgical tool 136. In other words, the surgical tool 136 may be gripped, held, or otherwise coupled to and controlled by the robotic arm 116. As such, the pose (e.g., position and orientation) of the surgical tool 136 may be controlled by the pose of the robotic arm 116. The surgical tool 136 can be controlled by one or more components of the system 100, such as the computing device 102. In some embodiments, the computing device 102 may be capable of receiving or retrieving data or other information (e.g., from the database 130, from one or more sensors, from the imaging device 112, etc.), process the information, and control the surgical tool 136 based on the processed information. Additionally or alternatively, the navigation system 118 may track the position of and/or navigate the surgical tool 136. Such tracking may enable the system 100 or components thereof (e.g., the computing device 102) to determine how the surgical tool 136 interacts with anatomical tissue and render updated depictions of the anatomical tissue to one or more displays (e.g., the navigation display 124) as discussed in further detail below.

The system 100 or similar systems may be used, for example, to carry out one or more aspects of the method 300 described herein. The system 100 or similar systems may also be used for other purposes.

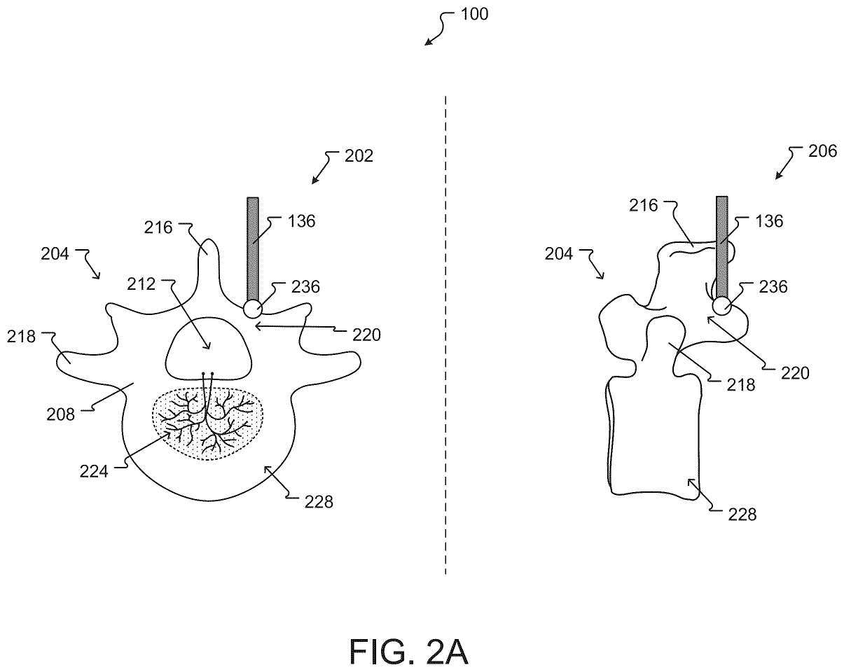

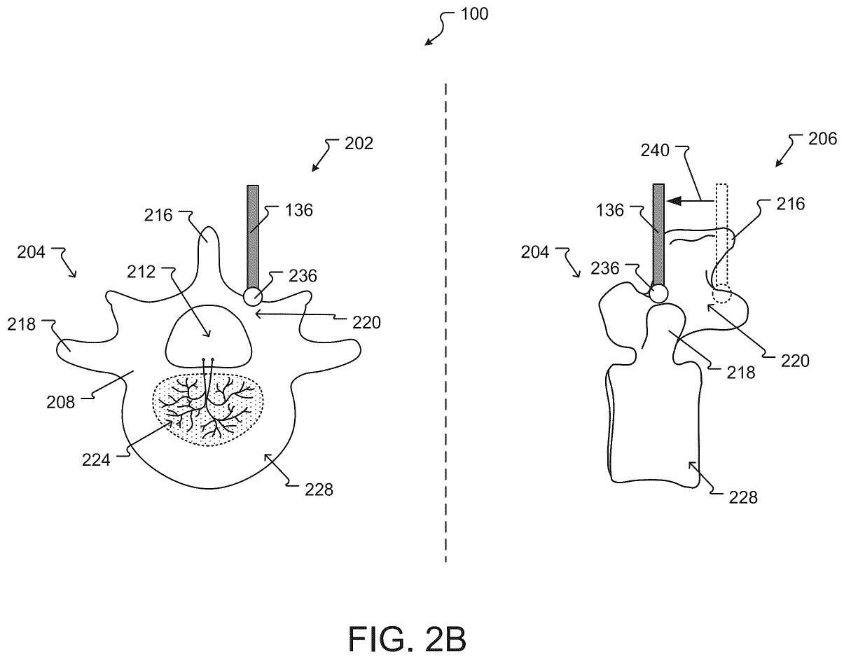

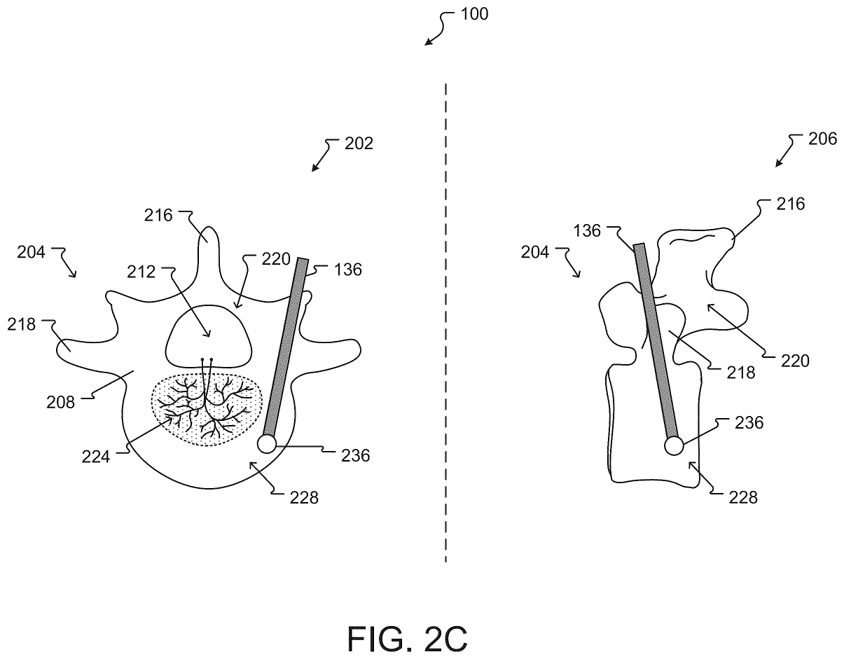

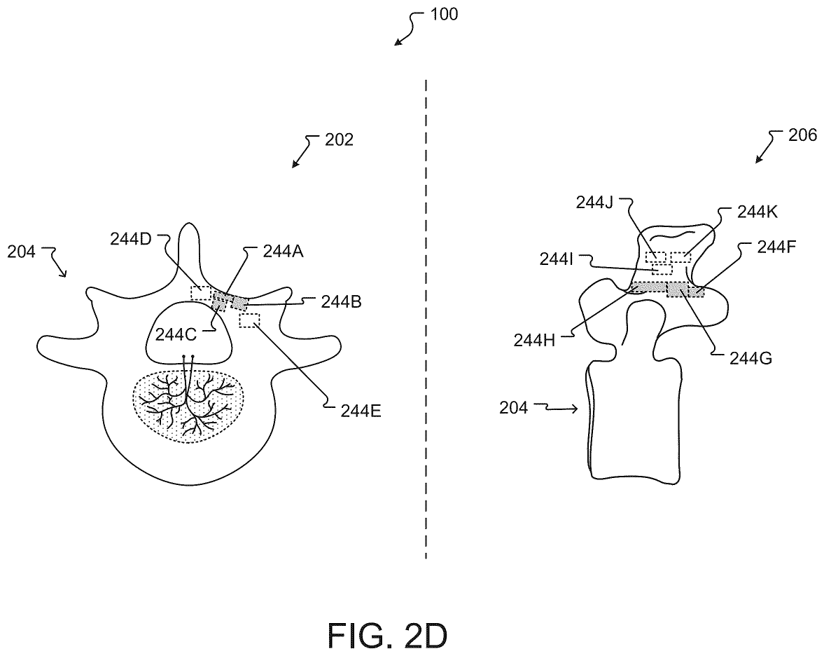

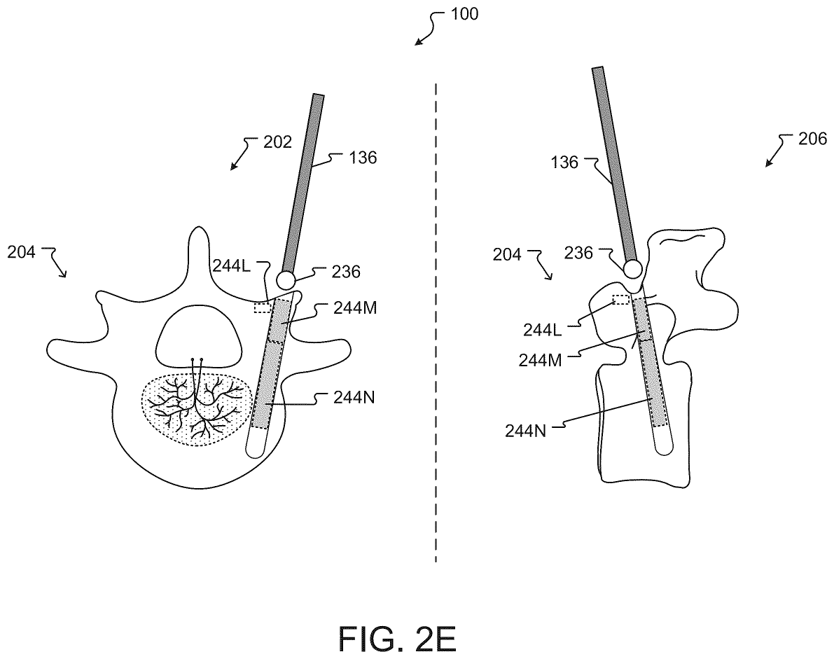

FIGS. 2A-2E depict aspects of a surgical tool 136 moving relative to a vertebra 204 according to at least one embodiment of the present disclosure. The movement of the surgical tool 136 relative to the vertebra 204 may occur when the surgery or surgical procedure comprises, for example, removing anatomical tissue from the vertebra 204. The anatomical tissue may be removed to form an autograft that can be used by a user (e.g., a surgeon) during the course of a spinal fusion surgery. In other cases, anatomical tissue may be removed to insert pedicle and/or cortical screws. In other cases, anatomical tissue may be removed to gain access to intervertebral disc space to perform disc decompression. It is to be understood that, while a vertebra 204 is depicted, in some examples the surgical tool 136 may interact with any other anatomical element (e.g., any other bone in the patient). The vertebra 204 comprises at least one pedicle 208, a vertebral foramen 212, a spinous process 216, a transverse process 218, lamina 220, nerves 224, and a vertebral body area 228.

With reference to FIGS. 2A-2E, each figure depicts a superior view 202 and a lateral view 206 of the vertebra 204. The superior view 202 may depict the vertebra 204 from the top of the patient (e.g., viewing the vertebra 204 while looking down on the patient's head), while the lateral view 206 may depict the vertebra 204 from a side of the patient (e.g., from the patient's right-hand side or from the patient's left-hand side).

Turning to FIG. 2A, a tool tip 236 of the surgical tool 136 is placed on or proximate to the vertebra 204. The tool tip 236 may be placed on any one or more portions of the outside surface of the vertebra 204, such as the at least one pedicle 208, the lamina 220, the spinous process 216, the transverse process 218, or the like. In one embodiment, the tool tip 236 may be placed on the lamina 220 of the vertebra 204. In some embodiments, the tool tip 236 may be placed elsewhere depending on the type of surgical tool or tool tip used, the type of surgery or surgical procedure, surgeon preference, combinations thereof, and the like. For example, when the surgical tool 136 comprises a drill to insert a pedicle screw, the tool tip 236 of the drill may be placed proximate the pedicle 208 so that the surgical tool 136 can drill down into the vertebra 204 to form a hole, as depicted in FIG. 2C. Then, another tool tip 236 may then be inserted into a drilled hole in the vertebra 204 to thread the hole for insertion of the pedicle screw.

The tool tip 236 may be or comprise an operational portion of the surgical tool 136 such as a drill, saw, cutter, reamer, burr, tap, or the like that enables the surgical tool to interact with the vertebra 204. For example, the surgical tool 136 may be or comprise a drill capable of drilling through bone, and the tool tip 236 comprises the surgical tip of the drill that can decorticate or resect anatomical tissue from the lamina 220 or facet of the vertebra 204. In another example, the surgical tool 136 may be or comprise an osteotome capable of cutting bone, and the tool tip 236 can decorticate or resect anatomical tissue from the lamina 220 or facet of the vertebra 204.

Information about the surgical tool 136 and/or the tool tip 236, as well as information about the surgical procedure (e.g., a spinal procedure) and/or the surgical workflow, may be stored in the database 130 and may be accessed during the course of the surgery or surgical procedure. The information may comprise information about the type, dimensions, and/or operating parameters of surgical tool 136 and/or the tool tip 236; information about whether or not the surgical tool 136 and/or the tool tip 236 is designed to decorticate or resect anatomical tissue; information about the surgical procedure and the surgical workflow; combinations thereof; and the like. Such information may be used, for example, by the navigation system 118 when tracking the surgical tool 136 to determine the locations on the vertebra 204 that interact with the surgical tool 136 and/or the tool tip 236 (e.g., to determine if the locations of the vertebra 204 have been resected). In another example, the navigation system 118 may use information about the surgical tool tip in conjunction with the current step in the surgical workflow to identify decorticated or resected anatomy. The information about the surgical tool 136 and/or the tool tip 236, information related to the navigation tracking of the surgical tool 136 and/or the tool tip 236 by the navigation system 118, one or more images of the vertebra 204, and/or information about the current step in a surgical workflow may be rendered to the navigation display 124.

Turning to FIGS. 2B-2C, aspects of the surgical tool 136 and the tool tip 236 interacting with the vertebra are shown in accordance with at least one embodiment of the present disclosure. As shown in FIG. 2B, the surgical tool 136 and the tool tip 236 may move across the vertebra 204 for the purposes of carrying out a surgical task performed during a surgery or surgical procedure. For example, the surgical tool 136 may comprise a drill, and the movement of the surgical tool 136 and the tool tip 236 across the vertebra 204 may occur when the surgical tool 136 is being used to resect anatomical tissues (e.g., bone) the vertebra 204, such as when a surgeon is gathering autograft to be used in a spinal fusion procedure. As another example and as depicted in FIG. 2C, the surgical tool 136 and the tool tip 236 may drill into a pedicle 208 of the vertebra 204 to create a hole into which a pedicle screw can be placed. While FIG. 2B depicts the tool tip 236 moving across the lamina 220 of the vertebra 204 in the direction of the arrow 240, and while FIG. 2C depicts the tool tip 236 moving into the pedicle 208 of the vertebra 204, it is to be understood that more generally the surgical tool 136 and/or the tool tip 236 may move across, move into, and/or interact with one or more other portions of the vertebra 204. For example, the surgical tool 136 and/or the tool tip 236 may interact with one or more facet joints of the vertebra 204, one or more spinous processes of the vertebra 204, one or more laminae of the vertebra 204, combinations thereof, and/or the like. Additionally or alternatively, the surgical tool 136 and/or the tool tip 236 may move across or interact with other vertebra, anatomical elements proximate the vertebra 204, or any other portion of patient anatomy.

The navigation system 118 may track the position of the surgical tool 136 and/or the tool tip 236 as the surgical tool 136 and the tool tip 236 interact with the vertebra 204. The navigation system 118 may use localizers (e.g., components that localize the location of the patient, the vertebra 204, the imaging device 112, etc. in a known coordinate space) and the imaging device 112 to track the position of the surgical tool 136 and/or the tool tip 236. In some embodiments, the surgical tool 136 may comprise navigation markers that can be tracked by the navigation system 118. In some embodiments, the tracking of the surgical tool 136 and/or the tool tip 236 may be rendered to the navigation display 124 for the user to view. For example, as the surgical tool 136 moves across the lamina 220, the navigation system 118 may render a visualization of the surgical tool 136 moving across a rendered visualization of the vertebra 204, such that the user can view in real-time or near real-time a depiction of the interaction between the vertebra 204 and the surgical tool 136.

FIGS. 2D-2E depict the vertebra 204 segmented into a plurality of voxels 244A-244N according to at least one embodiment of the present disclosure. In some examples, the depiction in FIG. 2D may correspond to the surgical maneuver depicted in FIG. 2B (where the tool tip 236 moves across the lamina 220 of the vertebra 204 in the direction of arrow 240), while the depiction in FIG. 2E may correspond to the surgical maneuver depicting in FIG. 2C (where the tool tip 236 moves into the pedicle 208 of the vertebra 204).

Each voxel of the plurality of voxels 244A-244N may be sections of an image depicting the vertebra 204 representative of a section (e.g., a 2D area or a 3D volume) of the vertebra 204 at that point in space. In other words, the image of the vertebra 204 may be segmented into the plurality of voxels 244A-244N, with each voxel of the plurality of voxels 244A-244N representing a portion of the vertebra 204 in 3D space (or, in some cases, in 2D space). In some embodiments, the plurality of voxels 244A-244N may cover the entirety of the image, while in other embodiments one or more portions of the image of the vertebra 204 may be segmented into voxels.

Each of the voxels includes an attenuation value. The attenuation value may reflect a propensity of the area (or volume) represented by the voxel to be penetrated by energy (e.g., radiation from an X-ray). In some embodiments, the attenuation value may be based on Hounsfield units (HU). Hounsfield units are dimensionless units universally used in CT scanning to express CT numbers in a standardized and convenient form. Hounsfield units are obtained from a linear transformation of measured attenuation coefficients. The transformations are based on the arbitrarily-assigned densities of air and pure water. For example, the radiodensity of distilled water at a standard temperature and pressure (STP) of zero degrees Celsius and 105 pascals is 0 HU; the radiodensity of air at STP is −1000 HU. While attenuation values of the voxel are discussed qualitatively (e.g., low attenuation, medium attenuation, high attenuation, etc.) and/or quantitatively (e.g., based on values in HU) herein, it is to be understood that that the values of the voxels discussed herein are in no way limiting.

Images of the vertebra 204 (e.g., an image depicting the superior view 202, an image depicting the lateral view 206, etc.) may be captured and segmented into the plurality of voxels 244A-244N. In some embodiments, the segmenting may be performed manually, with the user providing input (e.g., via the user interface 110) to create the plurality of voxels 244A-244N. Additionally or alternatively, the segmenting may be performed by the processor 104 using, for example, segmentation 122. The segmentation 122 may comprise one or more Artificial Intelligence (AI) and/or Machine Learning (ML) models or algorithms, such as a Convolutional Neural Networks (CNNs), Deep Neural Networks (DNNs), an autoencoder algorithm, a recurrent neural network (RNN) algorithm, and transformer neural network algorithm, a generative adversarial network (GAN) algorithm, linear regression, support vector machine (SVM) algorithm, random forest algorithm, hidden Markov model, and/or any combination thereof trained on data sets to segment an image of the vertebra 204 into the plurality of voxels 244A-244N. For example, in some embodiments, the at least one processor may be configured to utilize a combination of a CNN algorithm in conjunction with an SVM algorithm. For example, the segmentation 122 data model(s) may be trained on historical data sets of similar anatomical elements and/or similar surgeries or surgical procedures to identify one or more regions of interest and superimpose the plurality of voxels 244A-244N on the image of the vertebra 204. In some embodiments, the segmentation 122 may be semiautomatic, with the user capable of modifying the results of the segmentation 122 manually. In other words, the segmentation 122 may segment the image of the vertebra 204 and output a segmented image, and the user may be able to adjust the voxel dimensions in the segmented image, the position of one or more voxels of the plurality of voxels 244A-244N, combinations thereof, and the like manually via input into the user interface 110.

The segmentation 122 comprises labeling each voxel of the plurality of voxels 244A-244N as either having a first volume type or a second volume type. For example, voxels representing portions of the vertebra 204 such as a facet (e.g., the lamina 220, the spinous process 216, the transverse process 218, etc.) may be labeled as having the first volume type. Voxels representing portions of adipose tissue (e.g., tissue along the approach trajectory of the tool tip 236 to the vertebra 204) may in contrast may be labeled as having the second volume type. In such examples, the voxels with the first volume type may represent volumes of anatomical tissue that comprise bone, while the voxels with the second volume type may represent volumes of anatomical tissue that comprise non-bony tissue (e.g., fat). In some embodiments, the voxels may be labeled based on the attenuation values of the voxels. For example, bone has a greater attenuation value than fat due to the higher density of bone, so voxels that represent areas with high attenuation values (e.g., values above a predetermined threshold value stored in the database 130) may be labeled as having the first volume type, while voxels that represent areas with low attenuation values (e.g., values below the predetermined threshold value) may be labeled as having the second volume type.

Based on the tracking and the segmenting, the computing device 102 or components thereof (e.g., the processor 104) may determine which voxels of the plurality of voxels 244A-244N were occupied by the tool tip 236 when the tool tip 236 moved across the vertebra 204. For example, if the tool tip 236 moved across the lamina 220 in the direction of the arrow 240, the computing device 102 would determine that a first voxel 244A, a second voxel 244B, a third voxel 244C, a sixth voxel 244F, a seventh voxel 244G, and an eighth voxel 244H were all occupied by the tool tip 236. Additionally or alternatively, the computing device 102 may identify voxels of the plurality of voxels 244A-244N that were not occupied by or did not otherwise interact with the tool tip 236. For example, the computing device 102 may determine that a fourth voxel 244D, a fifth voxel 244E, a ninth voxel 244I, a tenth voxel 244J, and an eleventh voxel 244K did not interact with the tool tip 236. As another example and as depicted in FIG. 2E, if the tool tip 236 drilled into the pedicle 208 of the vertebra 204, the computing device 102 would determine that a twelfth voxel 244L was not occupied by the tool tip 236, but that a thirteenth voxel 244M and a fourteenth voxel 244N were occupied by the tool tip 236. In some embodiments, information from the computing device 102 about which voxels correspond to regions of the vertebra 204 that have interacted with the tool tip 236 and/or information about which voxels comprise the first volume type and/or the second volume type may be rendered to the navigation display 124 for the user (e.g., the surgeon) to see.

In some cases, the computing device 102 or components thereof (e.g., the processor 104) may only count a voxel as having interacted with the tool tip 236 when the voxel represents an area of the vertebra 204 that corresponds to bone. In other words, other areas of the segmented image that represent non-bony regions (e.g., voxels representing adipose tissue proximate the vertebra 204) may be excluded when determining which voxels interacted with the tool tip 236. In some embodiments, the computing device 102 may determine whether or not the region of the segmented image represents a bony or non-bony region based on HU values, and/or based on manual or automatic segmentation methods.

The computing device 102 may update the visual display of the navigation display 124 based on which voxels interacted with the tool tip 236, with the update including a change or modification to the visual indicator of one or more voxels. The displayed information on the navigation display 124 may depend on the type of surgical tool 136 that was used and tracked by the navigation system 118, surgical plan information, user input, combinations thereof, and/or the like. For example, when the surgical tool 136 comprises a pointer probe such as a navigated probe that is moved by the physician or other user when probing the vertebra 204, the computing device 102 may determine that no tissue has been removed, and may not consider the voxels through which the pointer probe has moved as being changed or modified. However, when the surgical tool 136 comprises a tool that resects or is capable of resecting anatomical tissue (e.g., a navigated drill), the computing device 102 may count the voxel through which the tool tip 236 passes as being changed or modified. In some embodiments, the computing device 102 may count those voxels with the first volume type through which the tool tip 236 as being changed or modified, while not counting voxels with the second volume type. In other words, the computing device 102 may not count voxels that have little or no bone content as being modified by the surgical tool 136.

As another example, the surgical tool 136 may comprise a drill that drills through the pedicle 208 of the vertebra 204 to create a pilot hole for a pedicle screw. The computing device 102 may use information associated with the drill (e.g., the trajectory of the drill with respect to the vertebra 204, the radius of the planned pilot hole, etc.) and the tracking of the drill by the navigation system 118 to determine which portions of the vertebra 204 have interacted with the surgical tool. As depicted in FIG. 2E, the computing device 102 may determine that the drill interacts with areas of the vertebra 204 represented by the thirteenth voxel 244M and the fourteenth voxel 244N, and may cause the visual depictions of the thirteenth voxel 244M and the fourteenth voxel 244N to be updated on the navigation display 124. The computing device 102 may also determine that the twelfth voxel 244L has not interacted with the tool tip 236, and may not change the visual depiction of the twelfth voxel 244L on the navigation display 124.

In another example, the surgical tool 136 may comprise an osteotome, in which case the tool tip 236 may be or comprise a blade. The computing device 102 may use information associated with the blade (e.g., the trajectory of the blade with respect to the vertebra 204, the width of the blade, etc.) when the blade is docked on the vertebra 204 to define a cutting plane. The computing device 102 may then define the smaller of the connected components separated by the plane to be changed or modified. In other words, the computing device 102 may expect that the volume of bone removed is smaller than the volume of the vertebra 204, and may identify the smaller voxel volume as being changed or modified.

Additionally or alternatively, the computing device 102 may update the visual display of the navigation display 124 based on information about the type of surgery or surgical procedure, information about the current step of the surgery or surgical procedure, combinations thereof, and/or the like. The computing device 102 may receive information from the database 130 such as the current step of the surgical workflow (which may include information about the current type of surgical tool in use), and use such information to determine whether or not interaction between the patient anatomy and the surgical tool warrants an update to the visual depiction of the patient anatomy on the navigation display 124. For example, the surgical procedure may include a step where a navigated probe is used on the vertebra 204. For this step, the computing device 102 may access the surgical workflow in the database 130, determine that the current step is a navigated probe step, and determine that the portions of the vertebra 204 that interact with the surgical tool 136 during this step have not been resected or decorticated. As a result, the visual depiction of the vertebra 204 on the navigation display 124 may remain unchanged during the navigated probe step. The workflow may further include another step where the surgical tool 136 drills through the pedicle 208 of the vertebra 204 to create a pilot hole for a pedicle screw. For this drilling step, the computing device 102 may access the surgical workflow in the database 130, determine that the current step is a drilling step, and determine that any portions of the vertebra that interact with the tool tip 236 of the surgical tool 136 during this step should be considered resected. Then, based on the tracking of the surgical tool 136 during the drilling step, the computing device 102 may update the depiction of the voxels on the navigation display 124 that represent the portions of the vertebra 204 that interacted with the surgical tool 136 during the drilling step to indicate the portions of the vertebra 204 have been resected.

The computing device 102 may update the depiction of voxels on the navigation display 124 that have been identified as corresponding to portions or sections of the vertebra 204 that interacted with the tool tip 236. The update may be or comprise a change in the rendered color of one or more of the voxels, a rendered border of one or more of the voxels, an addition of one or more visual labels indicating the state of the portion (e.g., “bone resected,” “bone decorticated,” etc.), combinations thereof, and/or the like. For example, when the surgical tool 136 comprises a drill that is used to drill a pilot hole for a pedicle screw, the voxels associated with the area of the vertebra 204 that is drilled into by the surgical tool 136 may be modified with an outline to indicate that the area of the vertebra 204 has been resected. As another example, when the surgical tool 136 comprises a drill that is used to decorticate a facet or a lamina of the vertebra 204, the voxels associated with the facet or lamina of the vertebra 204 may be rendered in a different color to indicate that the facet or lamina has been decorticated. The update of the voxel depiction on the navigation display 124 may include updated or modified versions of the voxels, which may indicate to the user that such sections of the vertebra 204 have been altered by interactions with the tool tip 236 of the surgical tool 136.

In some cases, the computing device 102 may update the visual depiction of the voxels based on input information, which may be based on inputs from the user (e.g., via the user interface 110). For example, the surgery or surgical procedure may include the surgeon performing an operation with the surgical tool 136. The user may perform the operation (e.g., drilling into the pedicle 208 for the later insertion of a pedicle screw), and may manually update the segmented image (e.g., by manipulating the voxels rendered to the navigation display 124, by inputting a command via the user interface 110 that the drilling step has been completed, etc.). Once the computing device 102 has received the input information, the computing device 102 may modify or otherwise update the depiction of the voxels accordingly.

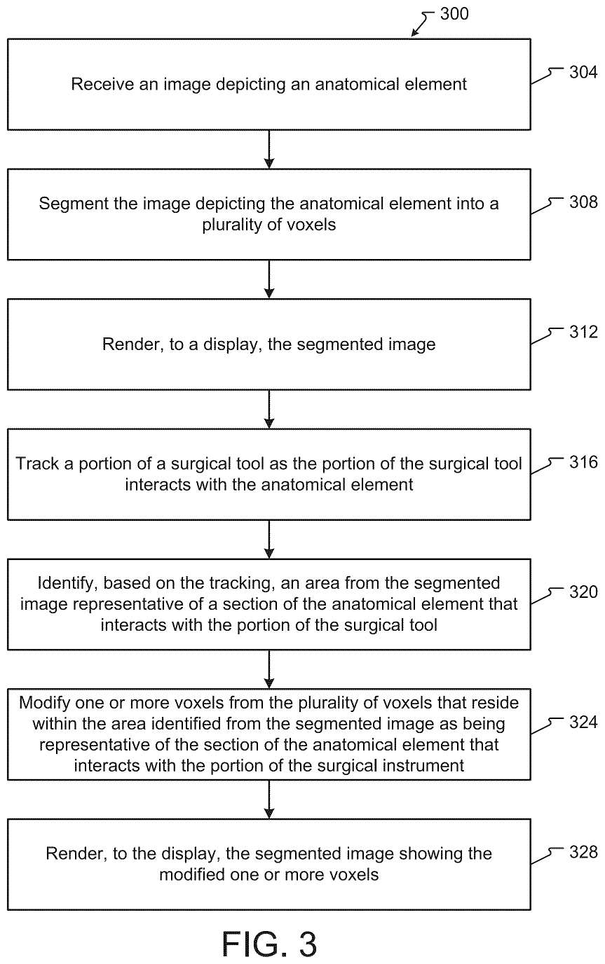

FIG. 3 depicts a method 300 that may be used, for example, to update a display based on changes to the state of an anatomical element during a surgery or surgical procedure.

The method 300 (and/or one or more steps thereof) may be carried out or otherwise performed, for example, by at least one processor. The at least one processor may be the same as or similar to the processor(s) 104 of the computing device 102 described above. The at least one processor may be part of a robot (such as a robot 114) or part of a navigation system (such as a navigation system 118). A processor other than any processor described herein may also be used to execute the method 300. The at least one processor may perform the method 300 by executing elements stored in a memory such as the memory 106. The elements stored in memory and executed by the processor may cause the processor to execute one or more steps of a function as shown in method 300. One or more portions of a method 300 may be performed by the processor executing any of the contents of memory, such as an image processing 120 and/or a segmentation 122.

The method 300 comprises receiving an image depicting an anatomical element (step 304). The image may be captured by the imaging device 112, and may depict the anatomical element that may be similar to or the same as the vertebra 204. In some embodiments, the image may depict additional anatomical elements, such as vertebrae adjacent to the vertebra 204. In some embodiments, the image may be captured during the course of a spinal fusion surgical procedure.

The method 300 also comprises segmenting the image depicting the anatomical element into a plurality of voxels (step 308). The plurality of voxels may be similar to or the same as the plurality of voxels 244A-244N. The segmenting may be performed by the processor 104 using, for example, segmentation 122. The segmentation 122 may comprise one or more data models (e.g., CNNs, DNNs, etc.) trained on data sets to receive an image or image data associated with the vertebra 204, segment the image of the vertebra 204 into the plurality of voxels 244A-244N, and output the segmented image to a display (e.g., to the navigation display 124). For example, the segmentation 122 data model(s) may be trained on historical data sets of similar anatomical elements and/or similar surgeries or surgical procedures to identify one or more regions of interest and superimpose the plurality of voxels 244A-244N on the image of the vertebra 204. In some embodiments, the segmentation 122 may be semiautomatic, with the user capable of modifying the results of the segmentation 122 manually. In other words, the segmentation 122 may segment the image of the vertebra 204, and the user may be able to adjust the segments, the position of one or more voxels of the plurality of voxels 244A-244N, combinations thereof, and the like manually via input into the user interface 110.

The method 300 also comprises rendering, to a display, the segmented image (step 312). The segmented image may be rendered to a display such as the navigation display 124 for the user (e.g., the surgeon) to see. In some cases, the user may be able to provide inputs into the display to alter, manipulate, or otherwise interact with the segmented image.

The method 300 also comprises tracking a portion of a surgical tool as the portion of the surgical tool interacts with the anatomical element (step 316). The surgical tool may be similar to or the same as the surgical tool 136. The tracking may be performed by the navigation system 118 tracking the surgical tool 136 (and/or the tool tip 236 of the surgical tool 136) using one or more navigation markers attached to the surgical tool 136. In such cases, the navigation system 118 may receive image data from the imaging device 112 that images the navigation markers on the surgical tool 136, and the navigation system 118 may use the processor 104 to determine the pose of the surgical tool 136 as well as changes thereto. Then, based on the movement of the surgical tool 136 relative to one or more localizers, the navigation system 118 may determine the pose and the change in pose of the surgical tool 136 relative to the vertebra 204.

The method 300 also comprises identifying, based on the tracking, an area from the segmented image representative of a section of the anatomical element that interacts with the portion of the surgical tool (step 320). Continuing from step 316, the computing device 102 may use pose information of the surgical tool 136 tracked by the navigation system 118 as the surgical tool 136 interacts with the vertebra 204 and the known pose of the vertebra 204 (e.g., based on navigation markers placed in known locations relative to the patient and registration between the navigation markers and the surgical tool 136) to determine which areas of the vertebra 204 interact with the tool tip 236 of the surgical tool 136. The computing device 102 may then determine which voxels of the plurality of voxels 244A-244N correspond to the area of the segmented image.

The method 300 also comprises modifying one or more voxels from the plurality of voxels that reside within the area identified from the segmented image as being representative of the section of the anatomical element that interacts with the portion of the surgical instrument (step 324). The computing device 102 may update the depiction of voxels on the navigation display 124 that have been identified as corresponding to portions or sections of the vertebra 204 that interacted with the tool tip 236. The update may be or comprise a change in the rendered color of one or more of the voxels, a rendered border of one or more of the voxels, an addition of one or more visual labels indicating the state of the portion (e.g., “bone resected,” “bone decorticated,” etc.), combinations thereof, and/or the like. For example, when the surgical tool 136 comprises a drill that is used to drill a pilot hole for a pedicle screw, the voxels associated with the area of the vertebra 204 that is drilled into by the surgical tool 136 may be modified with an outline to indicate that the area of the vertebra 204 has been resected. As another example, when the surgical tool 136 comprises a drill that is used to decorticate a facet or a lamina of the vertebra 204, the voxels associated with the facet or lamina of the vertebra 204 may be rendered in a different color to indicate that the facet or lamina has been decorticated. The update of the voxel depiction on the navigation display 124 may indicate to the user that such sections of the vertebra 204 have been modified.

In some cases, the computing device 102 may update the visual depiction of the voxels based on input information, which may be based on inputs from the user (e.g., via the user interface 110). For example, the surgery or surgical procedure may include the surgeon performing an operation with the surgical tool 136. The user may perform the operation (e.g., drilling into the pedicle 208 for the later insertion of a pedicle screw), and may manually update the segmented image (e.g., by manipulating the voxels rendered to the navigation display 124, by inputting a command via the user interface 110 that the drilling step has been completed, etc.). Once the computing device 102 has received the input information, the computing device 102 may modify or otherwise update the depiction of the voxels accordingly.

The method 300 also comprises rendering, to the display, the segmented image showing the modified one or more voxels (step 328). Once the voxels have been modified, the segmented image may be rendered to the display (e.g., the navigation display 124) as an updated segmented image that depicts the modified one or more voxels. The updated segmented image may enable the user to visualize the updated state of the vertebra 204, without the need for additional interoperative imaging.

The present disclosure encompasses embodiments of the method 300 that comprise more or fewer steps than those described above, and/or one or more steps that are different than the steps described above.

As noted above, the present disclosure encompasses methods with fewer than all of the steps identified in FIG. 3 (and the corresponding description of the method 300), as well as methods that include additional steps beyond those identified in FIG. 3 (and the corresponding description of the method 300). The present disclosure also encompasses methods that comprise one or more steps from one method described herein, and one or more steps from another method described herein. Any correlation described herein may be or comprise a registration or any other correlation.

The foregoing is not intended to limit the disclosure to the form or forms disclosed herein. In the foregoing Detailed Description, for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the foregoing has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

The techniques of this disclosure may also be described in the following examples.

Example 1: A system (100), comprising:
a processor (104); and
a memory (106) storing data thereon that, when processed by the processor (104), enable the processor (104) to:
receive an image depicting an anatomical element (204);
segment the image into a segmented image that includes a plurality of voxels (244A-244N);
track a portion (236) of a surgical instrument (136) as the portion (236) of the surgical instrument (136) interacts with the anatomical element (204);
identify, based on the tracking, an area from the segmented image representative of a section of the anatomical element (204) that interacts with the portion (236) of the surgical instrument (136);
modify one or more voxels from the plurality of voxels (244A-244N) that reside within the area identified from the segmented image as being representative of the section of the anatomical element (204) that interacts with the portion (236) of the surgical instrument (136); and
render, to a display (124), the segmented image showing the modified one or more voxels.

Example 2: The system according to example 1, wherein the one or more voxels are rendered with a first visual depiction a first time, and wherein the one or more voxels are rendered with a second visual depiction at a second time later than the first time.

Example 3: The system according to examples 1 or 2, wherein the modified one or more voxels are rendered with at least one of a different color and a different border than the plurality of voxels (244A-244N).

Example 4: The system according to any of examples 1 to 3, wherein the portion (236) of the surgical instrument (136) is capable of resecting anatomical tissue.

Example 5: The system according to any of examples 1 to 4, wherein the image is a two-dimensional image or a three-dimensional image.

Example 6: The system according to any of examples 1 to 5, wherein the tracking comprises determining a pose of the portion (236) of the surgical instrument (136) relative to the anatomical element (204) as the portion (236) of the surgical instrument (136) interacts with the anatomical element (204).

Example 7: The system according to any of examples 1 to 6, wherein the modified one or more voxels indicate that the portion of the anatomical element (204) has been resected.

Example 8: A system (100), comprising:
a processor (104); and
a memory (106) coupled with the processor (104) and storing data thereon that, when processed by the processor (104), enable the processor (104) to:
receive a segmented image depicting an anatomical element (204) segmented into a plurality of voxels (244A-244N);
render, to a display (124), the segmented image;
track a surgical tool (136) as the surgical tool (136) interacts with the anatomical element (204);
determine, based on the tracking, a voxel of the plurality of voxels (244A-244N) representative of a portion of the anatomical element (204) that interacts with the surgical tool (136); and
update a visual depiction of the voxel shown in the segmented image on the display (124).

Example 9: The system according to example 8, wherein the update of the visual depiction of the voxel comprises a change in at least one of a color and a border of the voxel.

Example 10: The system according to examples 8 or 9, wherein the update of the visual depiction of the voxel comprises an indicator that the portion of the anatomical element (204) has been resected.

Example 11: The system according to any of examples 8 to 10, wherein the segmented image comprises a two-dimensional image or a three-dimensional image, wherein the segmented image is received from an artificial intelligence data model that comprises a convolutional neural network that receives image data as an input and outputs the segmented image.

Example 12: The system according to any of examples 8 to 11, wherein the update of the visual depiction of the voxel is based on at least one of a type of surgical tool and a surgical workflow.

Example 13: A system (100), comprising:
a processor (104); and
a memory (106) coupled with the processor (104) and storing data thereon that, when processed by the processor (104), enable the processor (104) to:
receive image data associated with an anatomical element (204);
segment the image data into a plurality of voxels (244A-244N);
render, to a display (124), a visual depiction of the plurality of voxels (244A-244N);
track an operative portion (236) of a surgical instrument (136) as the operative portion (236) of the surgical instrument (136) interacts with the anatomical element (204);
identify, based on the tracking, a voxel of the plurality of voxels (244A-244N) associated with the operative portion (236) of the surgical instrument (136); and
render, based on the tracking, an updated visual depiction of the image data that includes a modified version of the voxel.

Example 14: The system according to example 13, wherein the operative portion (236) of the surgical instrument (136) is capable of resecting anatomical tissue, and wherein the modified version of the voxel provides an indicator that a section of the anatomical element (204) has been resected.

Example 15. The system according to examples 13 or 14, wherein the modified version of the voxel is rendered in the updated visual depiction with a first visual indicator when the surgical instrument (136) is a first type of surgical instrument, wherein the voxel is rendered with a second visual indicator when the surgical instrument (136) is a second type of surgical instrument, wherein the first visual indicator indicates that a section of the anatomical element (204) has been resected, and wherein the second visual indicator indicates that the section of the anatomical element (204) has been decorticated.

Various examples of the disclosure have been described. These and other examples are within the scope of the following claims.

What is claimed is:

1. A system, comprising:
a processor; and
a memory storing data thereon that, when processed by the processor, enable the processor to:
receive an image depicting an anatomical element;
segment the image into a segmented image that includes a plurality of voxels;
track a portion of a surgical instrument as the portion of the surgical instrument interacts with the anatomical element;
identify, based on the tracking, an area from the segmented image representative of a section of the anatomical element that interacts with the portion of the surgical instrument;
modify one or more voxels from the plurality of voxels that reside within the area identified from the segmented image as being representative of the section of the anatomical element that interacts with the portion of the surgical instrument; and
render, to a display, the segmented image showing the modified one or more voxels.

2. The system of claim 1, wherein the one or more voxels are rendered with a first visual depiction a first time, and wherein the one or more voxels are rendered with a second visual depiction at a second time later than the first time.

3. The system of claim 1, wherein the modified one or more voxels are rendered with at least one of a different color and a different border than the plurality of voxels.

4. The system of claim 1, wherein the portion of the surgical instrument is capable of resecting anatomical tissue.

5. The system of claim 1, wherein the image is a two-dimensional image or a three-dimensional image.

6. The system of claim 1, wherein the tracking comprises determining a pose of the portion of the surgical instrument relative to the anatomical element as the portion of the surgical instrument interacts with the anatomical element.

7. The system of claim 1, wherein the modified one or more voxels indicate that the portion of the anatomical element has been resected.

8. A system, comprising:
a processor; and
a memory coupled with the processor and storing data thereon that, when processed by the processor, enable the processor to:
receive a segmented image depicting an anatomical element segmented into a plurality of voxels;
render, to a display, the segmented image;
track a surgical tool as the surgical tool interacts with the anatomical element;
determine, based on the tracking, a voxel of the plurality of voxels representative of a portion of the anatomical element that interacts with the surgical tool; and
update a visual depiction of the voxel shown in the segmented image on the display.

9. The system of claim 8, wherein the update of the visual depiction of the voxel comprises a change in at least one of a color and a border of the voxel.

10. The system of claim 8, wherein the update of the visual depiction of the voxel comprises an indicator that the portion of the anatomical element has been resected.

11. The system of claim 8, wherein the segmented image comprises a two-dimensional image or a three-dimensional image.

12. The system of claim 8, wherein the segmented image is received from an artificial intelligence data model.

13. The system of claim 12, wherein the artificial intelligence data model comprises a convolutional neural network that receives image data as an input and outputs the segmented image.

14. The system of claim 8, wherein the tracking comprises determining a pose of the surgical tool relative to the anatomical element as the surgical tool interacts with the anatomical element.

15. The system of claim 8, wherein the update of the visual depiction of the voxel is based on at least one of a type of surgical tool and a surgical workflow.

16. A system, comprising:
a processor; and
a memory coupled with the processor and storing data thereon that, when processed by the processor, enable the processor to:
receive image data associated with an anatomical element;
segment the image data into a plurality of voxels;
render, to a display, a visual depiction of the plurality of voxels;
track an operative portion of a surgical instrument as the operative portion of the surgical instrument interacts with the anatomical element;
identify, based on the tracking, a voxel of the plurality of voxels associated with the operative portion of the surgical instrument; and
render, based on the tracking, an updated visual depiction of the image data that includes a modified version of the voxel.

17. The system of claim 16, wherein the operative portion of the surgical instrument is capable of resecting anatomical tissue.

18. The system of claim 17, wherein the modified version of the voxel provides an indicator that a section of the anatomical element has been resected.

19. The system of claim 16, wherein the modified version of the voxel is rendered in the updated visual depiction with a first visual indicator when the surgical instrument is a first type of surgical instrument, and wherein the voxel is rendered with a second visual indicator when the surgical instrument is a second type of surgical instrument.

20. The system of claim 19, wherein the first visual indicator indicates that a section of the anatomical element has been resected, and wherein the second visual indicator indicates that the section of the anatomical element has been decorticated.
